(19) United States
(12) Patent Application Publication
(10) Pub. No.: US 2021/0361353 A1
(43) Pub. Date: Nov. 25, 2021

(54) SYSTEMS AND METHODS FOR SENSING TEMPERATURE AT DIFFERENT LOCATIONS ON A MICROWAVE ANTENNA USING ULTIPLE FIBER SENSORS

(71) Applicant: Covidien LP, Mansfield, MA (US)

(72) Inventors: Leng Chen, Shanghai (CN); Wei Zhang, Shanghai (CN)

(21) Appl. No.: 16/637,359

(22) PCT Filed: Aug. 31, 2017
(86) PCT No.: PCT/CN2017/099878

§ 371 Date: Feb. 7, 2020

(51) Int. Cl. A61B 18/18

(52) U.S. Cl. CPC A61B 18/1815; A61B 2017/00057

(57) ABSTRACT

A microwave ablation system includes: a microwave generator (110) configured to generate a microwave power signal; a microwave antenna (120) coupled to the microwave field to heat at least a portion of the target tissue; a light source (312) for supplying an optical signal to multiple fiber sensors (320) disposed on the microwave antenna (120); a photodetector (316) for detecting an optical signal reflected from the fiber sensors (320) and producing an electrical detection signal; a demodulation device for demodulating the electrical detection signal; and a processor for processing the demodulated signal to obtain temperature measurements at different locations in the ablation zone, which may be displayed to a user to facilitate the performance of an ablation procedure. The multiple fiber sensors (320) may include multiple fiber Bragg gratings etched in an optical fiber (315), which are immune to electromagnetic interference.

### BACKGROUND

Microwave technology is commonly used to ablate tissues to treat diseases such as tumors. In microwave ablation procedures, the surgeon needs to know the real-time temperature inside the ablation zone, so as to track and monitor the status of the ablation area, and adjust the ablation power and time according to the real-time temperature. Usually a remote thermocouple needle is sold with an ablation antenna to detect the temperature outside the ablation zone. However, the thermocouple needle may not be used to detect the temperature inside the ablation zone because it may be affected by the microwave field surrounding the microwave antenna and may not give accurate temperature measurements. Moreover, a single thermocouple needle can only measure the temperature at a single point; thus, the single thermocouple needle is not suitable for use inside the ablation zone, in which the temperature may change significantly in different areas.

### SUMMARY

The present disclosure relates generally to an microwave ablation system with a fiber temperature sensor device. Fiber temperature sensors have many advantages including immunity to electromagnetic interference, lightweight, small size, and high sensitivity. In addition, one fiber temperature sensor including a plurality of Fiber Bragg Gratings (FBGs) can detect temperatures at multiple points in an ablation zone.

In aspects, the present disclosure features a microwave ablation system. The microwave ablation system includes a microwave generator having a microwave power source configured to generate a microwave power signal, a microwave antenna coupled to the microwave power source and configured to convert the microwave power signal to a microwave field to heat target tissue, and multiple fiber sensors disposed on the microwave antenna. The microwave ablation system further includes a light source that is coupled to the fiber sensors via an optical fiber and that transmits an optical signal to the fiber sensors, a photodetector that detects optical signals reflected from the fiber sensors, and a demodulation module that demodulates the reflected optical signal and determines multiple temperature measurements at different locations along at least a portion of the microwave antenna.

In aspects, the microwave ablation system may further include a microcontroller and the demodulation module may be implemented by instructions executed by the microcontroller.

In aspects, the microwave ablation system further includes an ablation pump that is in fluid communication with the microwave antenna and that supplies cooling fluid to the microwave antenna.

In aspects, the microwave ablation system further includes a display that displays the temperature measurements as a two-dimensional or three-dimensional map or profile.

In aspects, the microwave ablation system further includes an analog-to-digital converter that converts the electrical signal output from the photodetector into digital reflected signal data.

In aspects, the light source, the photodetector, and the demodulation module are incorporated into the microwave generator.

In aspects, the microwave ablation system includes a cable coupled between the microwave generator and the microwave antenna and includes a microwave transmission line and an optical fiber.

In aspects, the fiber sensors are fiber Bragg gratings etched in the optical fiber.

In aspects, the fiber sensors are disposed on the microwave antenna to correspond to a range of locations from a heated area of the target tissue to an unheated area of the target tissue.

In aspects, the fiber sensors are disposed in a distal portion of the optical fiber, and a fiber sensor of the fiber sensors is disposed between a proximal radiation portion and a distal radiating portion of the microwave antenna.

In aspects, the demodulation module performs wavelength division multiplexing, optical time domain reflectometry, optical frequency domain reflectometry, or code correlation to obtain the temperature measurements.

In aspects, the microwave ablation system includes an optical circulator coupled to the light source, the photodetector, and the fiber sensors.

In aspects, the microwave ablation system includes a reference reflector and an optical coupler having a first portion and a second portion. The first portion is coupled between the light source and the fiber sensors and the second portion is coupled between the photodetector and the reference reflector.

In aspects, the microwave ablation system includes a microcontroller in communication with the microwave generator and configured to control the microwave generator to adjust a characteristic of the microwave power signal and/or an ablation time based on the temperature measurements to control the size of a heated zone.

In aspects, the present disclosure also features a microwave instrument assembly. The microwave instrument assembly includes a cable including a microwave transmission line and an optical fiber, and a microwave antenna having a proximal radiating section and a distal radiating section. The microwave antenna is coupled to the cable, receives a microwave power signal via the microwave transmission line, and converts the microwave power signal to a microwave field surrounding at least a portion of the microwave antenna. The microwave instrument assembly also includes fiber sensors, which includes fiber Bragg gratings disposed in series on the microwave antenna, optically coupled to the optical fiber of the cable. A fiber Bragg grating of the plurality of fiber Bragg gratings is disposed between the proximal radiating section and the distal radiating section of the microwave antenna.

In aspects, the present disclosure also features a method of performing an ablation procedure. The method includes placing a microwave antenna having multiple fiber sensors disposed thereon in target tissue, transmitting microwave power to the microwave antenna to form a microwave field around at least a portion of the microwave antenna to heat target tissue, generating an optical interrogation signal, transmitting the optical interrogation signal to the fiber sensors, detecting an optical signal, generating an electrical detection signal, demodulating the electrical detection signal, processing the demodulated signal to obtain a plurality of temperature measurements at different locations within the ablation zone, and displaying the plurality of temperature measurements.

In aspects, the method may further include cooling the microwave antenna using a fluid based on the plurality of temperature measurements.

In aspects, the method may further include determining the locations of the plurality of temperatures in at least a portion of the ablation zone based on the electrical detection signal, generating a temperature profile based on the location of the temperature measurements, and displaying an ablation area a temperature profile throughout at least a portion of the ablation zone.

In aspects, the method may further include adjusting the level of the microwave power and/or the ablation time based on the plurality of temperature measurements to control the size of the ablation zone.

In aspects, the fiber sensors include fiber Bragg gratings.

In aspects, the method may further include demodulating the reflected optical signal by performing wavelength division multiplexing, optical time domain reflectometry, optical frequency domain reflectometry, or code correlation.

In aspects, detecting the optical signal includes detecting an interference signal between an optical signal reflected from the fiber sensors and an optical signal reflected from a reference reflector, and the method includes determining a location of each of the fiber sensors based on the interference signal.

### BRIEF DESCRIPTION OF THE DRAWINGS

Various aspects of the present disclosure are described below with reference to the drawings, wherein:

FIG. 1 is a block diagram illustrating a microwave ablation system according to embodiments of the present disclosure;

FIG. 2 is a cross-sectional view of a portion of a microwave antenna according to embodiments of the present disclosure;

FIG. 3 is a block diagram illustrating circuitry of the microwave generator of FIG. 1 according to embodiments of the present disclosure; and

FIG. 4 is a flow diagram illustrating an example process for performing an ablation procedure according to embodiments of the present disclosure.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the phrase “in embodiments” and variations on this phrase generally is understood to mean that the particular feature, structure, system, or method being described includes at least one iteration of the disclosed technology. Such phrase should not be read or interpreted to mean that the particular feature, structure, system, or method described is either the best or the only way in which the embodiment can be implemented. Rather, such a phrase should be read to mean an example of a way in which the described technology could be implemented, but need not be the only way to do so.

The microwave ablation systems of the present disclosure includes, among other components, the following components: a microwave generator with an optical fiber grating demodulation module; a microwave antenna with a fiber sensor disposed on the microwave antenna; and an ablation pump. The fiber sensor includes multiple sensing areas. In embodiments, the sensing areas are located along a portion of the length of the antenna from a highest temperature area to an unheated area. The highest temperature area may be located in the middle of the proximal radiating section (PRS) and the distal radiating section (DRS).

A microwave ablation procedure is conducted by first placing the microwave antenna into the target tissue. When the microwave generator is turned on, the generator transmits microwave power to the microwave antenna. The target tissue is heated by the microwave field formed by the antenna. The size of the heated zone can be controlled by both the magnitude of the power and the ablation time. Usually, there is also a cooling system using saline or de-ionized water to cool the antenna. During an ablation procedure, a fiber sensor, which includes multiple sensing areas and is disposed on the antenna, can detect the real-time temperatures inside the ablation zone and transmit an optical signal to the fiber grating demodulation module. After demodulation, the optical signal is converted into an electrical signal and then displayed to the user. The fiber sensor including multiple sensing areas greatly improves the user experience with the microwave ablation system. Instead of just following standard procedure, the surgeon can adjust the ablation power and time by themselves based on the real-time temperature a at different locations in the ablation zone.

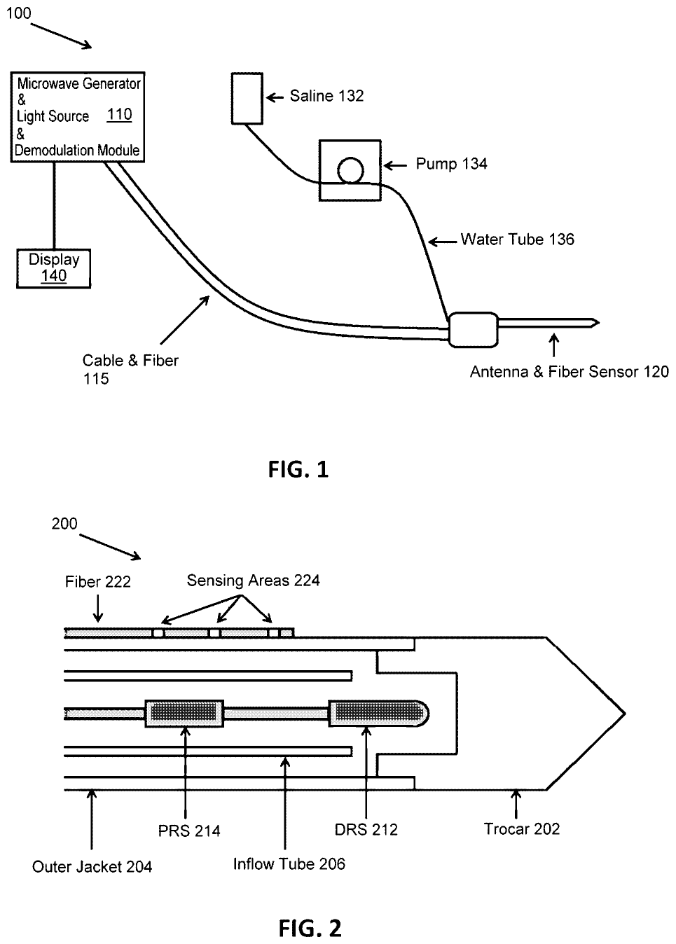

FIG. 1 is a block diagram illustrating a microwave ablation system 100 according to embodiments of the present disclosure. The microwave ablation system 100 includes a microwave generator 110 and a microwave antenna 120 coupled together via a cable 114. In embodiments, a fiber sensor including multiple sensing areas is disposed on the microwave antenna 120 and the cable 114 includes a microwave transmission line and an optical fiber. In embodiments, the microwave generator 110 includes a light source that transmits optical signals to the fiber sensor via the optical fiber. The microwave generator 110 also includes a demodulation module which receives an optical reflection signal from the fiber sensor and demodulates the optical reflection signal to obtain an electrical signal. The electrical signal is sampled and processed to obtain temperature measurements at different locations along at least a portion of the length of the microwave antenna.

The microwave ablation system 100 also includes a microwave antenna cooling system. The microwave antenna cooling system includes a saline source 132, a fluid pump 134, and a fluid tube 136. The fluid pump 134 pulls saline fluid from the saline source 132 and pushes saline fluid to the microwave antenna 120 to cool the microwave antenna 120 during a microwave ablation procedure.

FIG. 2 is a cross-sectional view of a portion 300 of the microwave antenna 120 of FIG. 1 according to embodiments of the present disclosure. The microwave antenna 120 includes a trocar 202 with a sharp tip at the distal portion of the microwave antenna 120 for penetrating tissue en route to the target tissue. The microwave antenna 120 also includes an outer jacket 204 on which fiber 222 having multiple sensing areas 224 is disposed. The microwave antenna 120 further includes a proximal radiating section (PRS) 214 and a distal radiating section (DRS) 212, which typically heat up the most during an ablation procedure. Thus, the microwave antenna 120 includes an inflow tube 206 for carrying cooling fluid to the PRS 214 and the DRS 212 to cool the PRS 214 and the DRS 212 when they heat up.

The sensing areas 224 for measuring temperatures may each include Fiber Bragg Gratings (FBGs). The FBGs may be etched in the fiber 222. The FBGs reflect a narrow wavelength range called the Bragg wavelength. Each FBG includes periodic modulations in the core of the fiber with spacing between each modulation. This changes the refractive index of the fiber 222 so that a single wavelength is reflected, while the rest of the light is transmitted down the fiber 222. The spacing between modulations changes when an FBG is subjected to a change in temperature. This changes the refractive index of the FBG and causes the Bragg wavelength to shift. Embodiments of the present disclosure use the shift in the Bragg wavelength to determine a temperature.

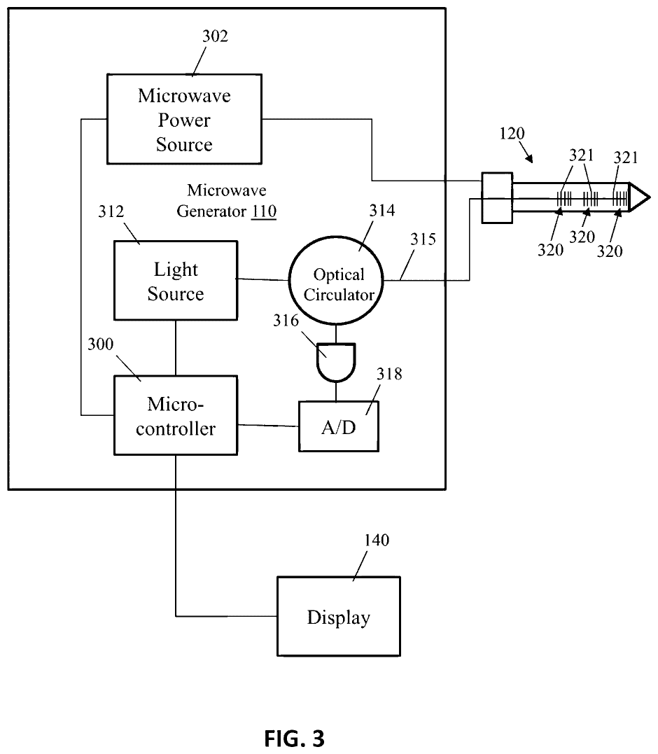

FIG. 3 is a block diagram illustrating circuitry of the microwave generator of FIG. 1 according to embodiments of the present disclosure. The microwave generator 110 includes a microcontroller 300 and a microwave power source 302 coupled to the microcontroller 300 so that the microcontroller 300 can control operation of the microwave power source 302. The microcontroller 300 may include one or more processing circuits, such as a central processing unit (CPU), a Field-Programmable Field Array (FPGA), or an Application-Specific Integrated Circuit (ASIC), and one or more memory structures, such as static random-access memory (SRAM), FLASH memory, and electrically erasable programmable read-only memory (EEPROM). The microcontroller 300 is configured to control the microwave power source 302 to generate a microwave power signal having an appropriate power level, which may be manually set by the user via a user control, e.g., a button (not shown), and/or which may be automatically set based on feedback parameters obtained, for example, by the fiber sensors 320 disposed on the microwave ablation antenna 120.

The microwave generator 110 also includes optical components which interface with the fiber sensors 320 via optical fiber 315 to measure temperature at different locations along a portion of the ablation instrument 120. The optical components include a light source 312, which may be a tunable light source and/or a broadband light source, an optical circulator 314, and a photodetector 316 optically coupled to each other. In embodiments, the light source may be tuned to different frequencies which correspond to different fiber sensors 320. The microwave generator 110 further includes an analog-to-digital (A/D) converter 318 electrically coupled to the photodetector 316. The light source 312 generates and transmits light signals to the optical sensors 320 via the optical circulator 315 and the optical fiber 315. The light source 312 may be a semiconductor or solid state laser.

The optical circulator 313 receives reflected light via the optical fiber 315 and directs the reflected light to the photodetector 316. The photodetector 316 converts the reflected light into an electrical signal, which is converted into digital data by the A/D converter 318. The microcontroller 300 then processes the digital data to obtain temperature measurements. The processing may include performing a Fourier transform on the digital data output from the A/D converter 318.

The fiber 315 includes a core, a cladding disposed over the core, and a buffer coating covering the cladding. The distal portion of the fiber 315 includes multiple Fiber Bragg Gratings (FBGs) 320. The FBGs 320 may be etched into the fiber 300 and/or the FBGs 320 may be configured to have different wavelengths. This is useful for using a single fiber 300 to sense temperature at multiple locations along the ablation probe 120. In further embodiments, multiple fibers 300 may be included each having one or more gratings 320.

The FBGs 320 include a plurality of reflection points 321 written into the optical fiber 315 at periodic spacing “Λ.” In some embodiments, the FBGs 320 may be written into the optical fiber 315 using high intensity pulses from a laser (e.g., argon fluoride excimer laser with a phase mask). As the optical fiber 315 undergoes mechanical strain (e.g., a change in length) due to temperature and pressure changes, the spacing Λ is modified due to stretching or contraction of the fiber 315. The effects of temperature on the fiber is quantified by the microcontroller 300 by measuring the wavelength shift in light reflected by the reflection points 321 based on the following equation:

$$\frac{Δ λ}{{λ}_{0}} = k * ɛ + {α}_{δ} * Δ T \qquad ( 1 )$$

In equation (1), Δλ is the wavelength shift, λ0 is the base wavelength, k is a gage factor, which is a difference between 1 and a photo-elastic coefficient, ρ, ε is strain, ΔT is a temperature change, and αδ is a change of the refraction index.

In this manner, fiber 315 is configured to transmit at least one wavelength of light and the FBGs 320 are configured to reflect at least one wavelength of light. Thus, the light transmissive properties, namely transmittance, of the distal portion of the fiber 315 corresponds to a set of physical parameters of the microwave antenna 120. The light transmissive properties of the distal portion of the optical fiber 315 may transition with changing physical parameters or a changed condition of the microwave antenna 120. In particular, changes in temperature and pressure affect (e.g., stretch or contract) the distal portion of the optical fiber 315, which, in turn, modifies the spacing between the FBGs 320. The changes in spacing between the gratings 320 changes the wavelength of the light reflected back through the fiber 315. The change in the wavelength is then used by the microcontroller 300 to determine the change in temperatures at the locations of the gratings 320.

The microcontroller 300 includes a fiber grating demodulator, which demodulates the reflected light using a demodulation technique to obtain the changes in wavelength. Demodulation techniques include wavelength division multiplexing (WDM), optical time domain reflectometry (OTDM), optical frequency domain reflectometry (OFDM), and code correlation techniques that incorporate aspects of OTDM and OFDM. According to the OTDR technique, a narrow light pulse is generated by the light source 312 and is transmitted through the optical fiber 315 to the fiber Bragg gratings 320. The reflected or backscattered light is analyzed to determine multiple temperatures. The locations corresponding to each of the temperatures may be determined by monitoring the time it takes the reflected or backscattered light to return to the photodetector 316.

According to the OFDR technique, the reflected or backscattered light is measured as a function of frequency, and then is subjected to a Fourier transformation. In OFDR, the light source may employ a quasi-continuous wave mode and the photodetector 316 performs narrow-band detection of the reflected or backscattered light.

According to the code correlation technique, the light source 312 transmits on/off sequences, which represent codes, into the optical fiber 315. The codes are chosen to have appropriate properties and the optical energy is spread over the codes. The detected reflected or backscattered light is transformed into a spatial profile, e.g., by cross-correlation, and is analyzed to determine a temperature profile corresponding to the spatial profile.

In other embodiments, the optical circulator 314 may be replaced with an optical coupler which optically couples the optical fiber 315 between the light source 312 and the fiber bragg gratings 320 to an optical fiber (not shown) between the photodetector 316 and a reference detector (not shown). In operation, the light reflected from the reference reflector interferes with the light reflected from the Fiber Bragg Gratings (FBGs) 320. The photodetector 316 detects the interference and produces a detector current signal that has a phase term dependent on the distances between the reference reflector and the FBGs 320. Because the frequency of the detector current signal is proportional to the distance from the reference reflector, a Fourier transform of the detector current signal results in the locations of each FBG 320. Based on the locations of the FBGs 320, the detector signal may be passed through narrow-band frequency filters to separate out spectral information from individual FBGs 320. The spectral information is then processed to obtain temperature measurements at the locations of the FBGs 320.

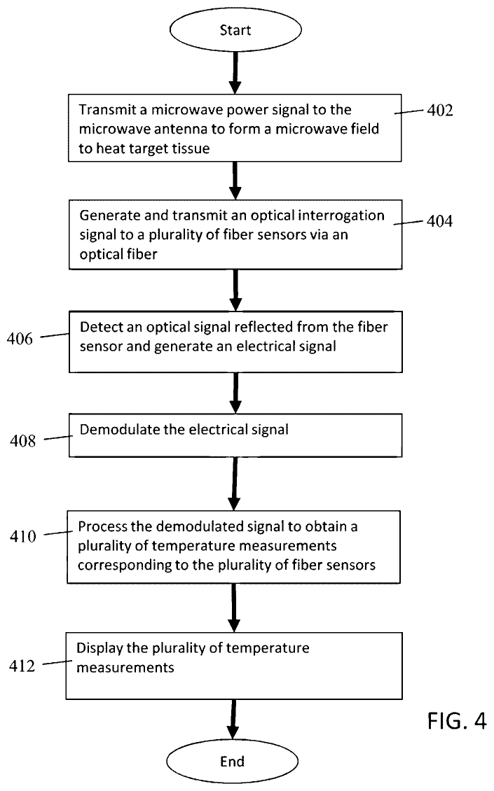

FIG. 4 is a flow diagram illustrating an example process for performing an ablation procedure according to embodiments of the present disclosure. When a microwave ablation procedure is conducted, the microwave antenna is placed in target tissue. When the microwave generator is turned on, the generator transmits microwave power to the microwave antenna to form a microwave field to heat target tissue at block 402. At block 404, an optical interrogation signal is generated and transmitted to fiber sensors or FBGs via an optical fiber. At block 406, an optical signal reflected from the fiber sensors is detected and an electrical detection signal based on the reflected optical signal is generated. At block 408, the electrical detection signal is demodulated, and, at block 410, the demodulated signal is processed to obtain multiple temperature measurements corresponding to the FBGs. At block 412, the temperature measurements are displayed. In embodiments, the temperature measurements may be used to create a two-dimensional or three-dimensional temperature map or temperature profile, which may be displayed.

For example, the two-dimensional temperature profile may be a graph having an axis for temperature and an axis for location along the microwave ablation antenna. As another example, the two-dimensional temperature map may show temperatures in a plane intersecting the central axis of the microwave ablation antenna, where the x-axis of the plane is the central axis of the microwave ablation antenna and the y-axis of the plane is perpendicular to the central axis of the microwave ablation antenna. The temperatures along the x-axis may be the temperature measurements corresponding to the fiber sensors. And the temperatures along the y-axis may be temperature estimates based on the temperature measurements, the perpendicular distance from the x-axis, and a model of the tissue in which the microwave antenna is disposed. Temperatures may be shown by a range of colors or by multiple numeric indications of the temperature distributed throughout the plane.

In embodiments, the three-dimensional temperature map may show temperatures in a volume intersecting the central axis of the microwave ablation antenna, where the x-axis of the volume is the central axis of the microwave ablation antenna and the y-axis and the z-axis of the volume are perpendicular to the central axis of the microwave ablation antenna. A three-dimensional map may be constructed in a manner similar to the two-dimensional map. The temperatures along the x-axis may be the temperature measurements corresponding to the fiber sensors. And the temperatures along the y-axis and the z-axis may be temperature estimates based on the temperature measurements, the perpendicular distance from the x-axis, and a model of the tissue in which the microwave antenna is disposed.

In embodiments, the two-dimensional profile may show the measured temperatures on a graph having an x-axis representing location on the microwave antenna and a y-axis representing time.

Using the displayed real-time temperature measurements, a surgeon performing an ablation procedure can more accurately control the heating of the target tissue by viewing the temperature measurements at different locations in the ablation zone and manually adjusting the microwave power level and/or the ablation time to control the size of the heated zone. In other embodiments, the level of the microwave power and/or the ablation time may be automatically adjusted by the microcontroller 300 based on the multiple temperature measurements to control the size of a desired heated zone, e.g., by transmitting control signals to the microwave power source 302.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope and spirit of the claims appended hereto. For example, while the present disclosure makes reference to a particular optical system for measuring temperature using a fiber sensor, the present disclosure contemplates other optical systems suitable for detecting an optical signal reflected from an optical sensor, demodulating the signal, and determining temperature measurements based on the demodulated signal.

1. A microwave ablation system, comprising:
a microwave generator having a microwave power source configured to generate a microwave power signal;
a microwave antenna coupled to the microwave power source and configured to convert the microwave power signal to a microwave field to heat target tissue;
a plurality of fiber sensors disposed on the microwave antenna;
a light source coupled to the plurality of fiber sensors via an optical fiber and configured to transmit an optical signal to the plurality of fiber sensors;
a photodetector configured to detect an optical signal reflected from the plurality of fiber sensors; and
a demodulation module configured to demodulate the detected optical signal and to determine a plurality of temperature measurements at different locations along the microwave antenna.

2. The microwave ablation system of claim 1, further comprising a microcontroller, wherein the demodulation module is implemented by a plurality of instructions executed by the microcontroller.

3. The microwave ablation system of claim 2, further comprising an ablation pump in fluid communication with the microwave antenna and configured to supply cooling fluid to the microwave antenna,
wherein the microcontroller is configured to control the ablation pump based on the plurality of temperature measurements.

4. The microwave ablation system of claim 1, further comprising a display configured to display the plurality of temperature measurements as a two-dimensional or three-dimensional map or profile.

5. The microwave ablation system of claim 1, further comprising an analog-to-digital converter configured to convert the electrical signal output from the photodetector into digital reflected signal data.

6. The microwave ablation system of claim 1, wherein the light source, the photodetector, and the demodulation module are incorporated into the microwave generator,
further comprising a cable coupled between the microwave generator and the microwave antenna and including a microwave transmission line and the optical fiber.

7. The microwave ablation system of claim 1, wherein the plurality of fiber sensors are a plurality fiber Bragg gratings etched in the optical fiber.

8. The microwave ablation system of claim 1, wherein the plurality of fiber sensors are disposed on the microwave antenna to correspond to a range of locations from a heated area of the target tissue to an unheated area of the target tissue.

9. The microwave ablation system of claim 1, wherein the plurality of fiber sensors are disposed in the optical fiber, and
wherein a fiber sensor of the plurality of fiber sensors is disposed between a proximal radiation portion and a distal radiating portion of the microwave antenna.

10. The microwave ablation system of claim 1, wherein the demodulation module performs wavelength division multiplexing, optical time domain reflectometry, optical frequency domain reflectometry, or code correlation to obtain the plurality of temperature measurements.

11. The microwave ablation system of claim 1, further comprising an optical circulator coupled to the light source, the photodetector, and the plurality of fiber sensors.

12. The microwave ablation system of claim 1, further comprising a reference reflector; and
an optical coupler having a first portion and a second portion, the first portion coupled between the light source and the plurality of fiber sensors, the second portion coupled between the photodetector and the reference reflector.

13. The microwave ablation system of claim 1, further comprising a microcontroller in communication with the microwave generator and configured to control the microwave generator to adjust a characteristic of the microwave power signal and/or an ablation time based on the plurality of temperature measurements to control the size of a heated zone.

14. A microwave instrument assembly, comprising:
a cable including a microwave transmission line and an optical fiber;
a microwave antenna having a proximal radiating section and a distal radiating section, the microwave antenna coupled to the cable, configured to receive a microwave power signal via the microwave transmission line, and configured to convert the microwave power signal to a microwave field surrounding at least a portion of the microwave antenna; and
a plurality of fiber Bragg gratings disposed in series on the microwave antenna and coupled to the optical fiber of the cable, a fiber Bragg grating of the plurality of fiber Bragg gratings disposed between the proximal radiating section and the distal radiating section.

15. A method of performing an ablation procedure, comprising:
transmitting a microwave power signal to a microwave antenna to form a microwave field around at least a portion of the microwave antenna to heat target tissue;
generating an optical interrogation signal;
transmitting the optical interrogation signal to a plurality of fiber sensors;
detecting an optical signal and generating an electrical detection signal;
demodulating the electrical detection signal;
processing the demodulated signal to obtain a plurality of temperature measurements at different locations within an ablation zone; and
displaying the plurality of temperature measurements.

16. The method of claim 15, further comprising cooling the microwave antenna using a fluid based on the plurality of temperature measurements.

17. The method of claim 15, further comprising:
determining the locations of the plurality of temperatures in at least a portion of the ablation zone based on the electrical detection signal;
generating a temperature profile based on the location of the plurality of temperature measurements; and
displaying an ablation zone and a temperature profile throughout at least a portion of the ablation zone.

18. The method of claim 15, further comprising adjusting the level of the microwave power signal and/or an ablation time based on the plurality of temperature measurements to control the size of a heated zone.

19. The method of claim 15, wherein the plurality of fiber sensors includes a plurality of fiber Bragg gratings.

20. The method of claim 15, further comprising demodulating the detected optical signal by performing wavelength division multiplexing, optical time domain reflectometry, optical frequency domain reflectometry, or code correlation.

21. The method of claim 15, wherein detecting the optical signal includes detecting an interference signal between an optical signal reflected from the plurality of fiber sensors and an optical signal reflected from a reference reflector,
further comprising determining a location of each of the plurality of fiber sensors based on the interference signal.
